(19) United States
(12) Patent Application Publication
(10) Pub. No.: US 2004/0148653 A1
(43) Pub. Date: Jul. 29, 2004

(54) Triacylglycerol lipases

(76) Inventors: Edgar B. Cahoon, Webster Groves, MO (US); Rebecca E. Cahoon, Webster Groves, MO (US); Anthony J. Kinney, Wilmington, DE (US); J. Antoni Rafalski, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/786,490

(22) Filed: Feb. 25, 2004

Related U.S. Application Data
(62) Division of application No. 09/699,652, filed on Oct. 30, 2000, now abandoned.
Application No. 09/699,652 is a continuation of application No. PCT/US99/09280, filed on Apr. 29, 1999.
(60) Provisional application No. 60/083,688, filed on Apr. 30, 1998.

(51) Int. Cl. A01H 1/00; C12N 15/82; C12Q 1/68; C07H 21/04; C12N 9/20; C12N 5/04

(52) U.S. Cl. 800/281; 435/6; 435/69.1; 435/320.1; 435/419; 435/198; 536/23.2

(57) ABSTRACT

This invention relates to an isolated nucleic acid fragment encoding a triacylglycerol lipase. The invention also relates to the construction of a chimeric gene encoding all or a portion of the triacylglycerol lipase, in sense or antisense orientation, wherein expression of the chimeric gene results in production of altered levels of the triacylglycerol lipase in a transformed host cell.

This application is a divisional of U.S. application Ser. No. 09/699,652, filed Oct. 30, 2000 which is a continuation of International Application No. PCT/US99/09280, filed Apr. 29, 1999 now expired, which claims the benefit of U.S. Provisional Application No. 60/083,688, filed Apr. 30, 1998 now expired. The entire content of these applications is herein incorporated by reference in their entirety.

### FIELD OF THE INVENTION

This invention is in the field of plant molecular biology. More specifically, this invention pertains to nucleic acid fragments encoding triacylglycerol lipases in plants and seeds.

### BACKGROUND OF THE INVENTION

True lipases attach triacylglycerols and act at an oil-water interface; they constitute a ubiquitous group of enzymes catalyzing a wide variety of reactions, many with industrial potential. Triacylglycerol lipases catalyze the transformation of triacylglycerol and water into diacylglycerol and a fatty acid anion. Human gastric lipase, rat lingual lipase, and human hepatic lysosomal lipase amino acid sequences are homologous but are unrelated to porcine pancreatic lipase apart from a 6 amino-acid sequence around the essential Ser-152 of porcine pancreatic lipase (Bodmer, M. W. (1987) Biochim Biophys Acta 909:237-244). These enzymes are glycosylated, contain a hydrophobic signal peptide, and belong to a gene family of acid lipases (Ameis, D. et al. (1994) Eur J Biochem 219:905-914). Lysosomal acid lipase (LAL) is a hydrolase essential for the intracellular degradation of cholesteryl esters and triacylglycerols and participates in the mobilization of seed oil during germination. No plant triacylglycerol lipase cDNAs of this class are currently listed in GenBank.

Neutral triacylglycerol lipases have been widely studied in fungi, bacteria, mammals, and insects. Nucleotide sequences with similarities to neutral triacylglycerol lipases in Arabidopsis thaliana and Ipomea nil have been described but their function has not yet been proven. The X-ray structure of the Mucor miehei triglyceride lipase has been reported, revealing a Ser . . . His . . . Asp trypsin-like catalytic triad with an active serine buried under the short helical fragment of a long surface loop (Brady, L. et al. (1990) Nature 343:767-770).

It may be useful to isolate triacylglycerol lipase cDNAs from plants that accumulate large amounts of fatty acids with unusual structures. Lacking this ability could be a possible limitation in development of transgenic crops with novel seed oils. Triacylglycerol lipases may also be useful in processing of plant seed oils. Lysosomal acid lipase (LAL) may be used to engineer lipid and cholesteryl ester metabolism and/or lysosome function.

### SUMMARY OF THE INVENTION

The instant invention relates to isolated nucleic acid fragments encoding triacylglycerol lipases. Specifically, this invention concerns an isolated nucleic acid fragment encoding an acid or a neutral triacylglycerol lipase. In addition, this invention relates to a nucleic acid fragment that is complementary to the nucleic acid fragment encoding an acid or a neutral triacylglycerol lipase.

An additional embodiment of the instant invention pertains to a polypeptide encoding all or a substantial portion of a triacylglycerol lipase selected from the group consisting of acid and neutral triacylglycerol lipases.

In another embodiment, the instant invention relates to a chimeric gene encoding an acid or a neutral triacylglycerol lipase, or to a chimeric gene that comprises a nucleic acid fragment that is complementary to a nucleic acid fragment encoding an acid or a neutral triacylglycerol lipase, operably linked to suitable regulatory sequences, wherein expression of the chimeric gene results in production of levels of the encoded protein in a transformed host cell that is altered (i.e., increased or decreased) from the level produced in an untransformed host cell.

In a further embodiment, the instant invention concerns a transformed host cell comprising in its genome a chimeric gene encoding an acid or a neutral triacylglycerol lipase, operably linked to suitable regulatory sequences. Expression of the chimeric gene results in production of altered levels of the encoded protein in the transformed host cell. The transformed host cell can be of eukaryotic or prokaryotic origin, and include cells derived from higher plants and microorganisms. The invention also includes transformed plants that arise from transformed host cells of higher plants, and seeds derived from such transformed plants.

An additional embodiment of the instant invention concerns a method of altering the level of expression of an acid or a neutral triacylglycerol lipase in a transformed host cell comprising: a) transforming a host cell with a chimeric gene comprising a nucleic acid fragment encoding an acid or a neutral acid triacylglycerol lipase; and b) growing the transformed host cell under conditions that are suitable for expression of the chimeric gene wherein expression of the chimeric gene results in production of altered levels of acid or neutral triacylglycerol lipase in the transformed host cell.

An addition embodiment of the instant invention concerns a method for obtaining a nucleic acid fragment encoding all or a substantial portion of an amino acid sequence encoding an acid or a neutral triacylglycerol lipase.

### BRIEF DESCRIPTION OF THE DRAWINGS AND SEQUENCE DESCRIPTIONS

The invention can be more fully understood from the following detailed description and the accompanying drawings and Sequence Listing which form a part of this application.

FIG. 1A-C depicts the amino acid sequence alignment between the acid triacylglycerol lipase from rice clone rlr72.pk0015.b2 (SEQ ID NO:14), soybean contig assembled from clones sdp3c.pk004.n3 and ssl.pk0022.a1 (SEQ ID NO:18), soybean contig assembled from clones sls1c.pk009.o2, srr1c.pk001.m19 and sre.pk0004.d7 (SEQ ID NO:20), Canis familiaris (NCBI General Identifier No. 3041702, SEQ ID NO:35) and Caenorhabditis elegans (NCBI General Identifier No. 3165581, SEQ ID NO:36). Amino acids which are conserved among all sequences are indicated with an asterisk (*) while amino acids conserved only among plant sequences are indicated by a plus sign (+). Dashes are used by the program to maximize alignment of the sequences. FIG. 1A, amino acids 1-180; FIG. 1B, amino acids 181-360; FIG. 1C, amino acids 361-433.

The following sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence comprising the entire cDNA insert in clone cen3n.pk0129.e9 encoding a portion of a corn acid triacylglycerol lipase.

SEQ ID NO:2 is the deduced amino acid sequence of a portion of a corn acid triacylglycerol lipase derived from the nucleotide sequence of SEQ ID NO:1.

SEQ ID NO:3 is the nucleotide sequence comprising the 3′ 647 nucleotides from the cDNA insert in clone ncs.pk0013.h1 encoding the C-terminal quarter of a Catalpa acid triacylglycerol lipase

SEQ ID NO:4 is the deduced amino acid sequence of the C-terminal quarter of a Catalpa acid triacylglycerol lipase derived from the nucleotide sequence of SEQ ID NO:3.

SEQ ID NO:5 is the nucleotide sequence comprising the 5′ 705 nucleotides from the cDNA insert in clone ncs.pk0013.h1 encoding the N-terminal third of a Catalpa acid triacylglycerol lipase.

SEQ ID NO:6 is the deduced amino acid sequence of the N-terminal third of a Catalpa acid triacylglycerol lipase derived from the nucleotide sequence of SEQ ID NO:5.

SEQ ID NO:7 is the nucleotide sequence comprising the contig assembled from a portion of the cDNA insert in clones p0075.cslag33r, p0126.cnlay46r and p0014.ctuty54r encoding a substantial portion of a corn acid triacylglycerol lipase.

SEQ ID NO:8 is the deduced amino acid sequence of a substantial portion of a corn acid triacylglycerol lipase derived from the nucleotide sequence of SEQ ID NO:7.

SEQ ID NO:9 is the nucleotide sequence comprising a portion of the cDNA insert in clone p0102.ceral64r encoding a portion of a corn acid triacylglycerol lipase.

SEQ ID NO:10 is the deduced amino acid sequence of a portion of a corn acid triacylglycerol lipase derived from the nucleotide sequence of SEQ ID NO:9.

SEQ ID NO:11 is the nucleotide sequence comprising a portion of the cDNA insert in clone p0126.cnlcm37r encoding a portion of a corn acid triacylglycerol lipase.

SEQ ID NO:12 is the deduced amino acid sequence of a portion of a corn acid triacylglycerol lipase derived from the nucleotide sequence of SEQ ID NO:11.

SEQ ID NO:13 is the nucleotide sequence comprising the entire cDNA insert in clone rlr72.pk0015.b2 encoding an entire rice acid triacylglycerol lipase.

SEQ ID NO:14 is the deduced amino acid sequence of an entire rice acid triacylglycerol lipase derived from the nucleotide sequence of SEQ ID NO:13.

SEQ ID NO:15 is the nucleotide sequence comprising a portion of the cDNA insert in clone rsl1n.pk012.h7 encoding a portion of a rice acid triacylglycerol lipase.

SEQ ID NO:16 is the deduced amino acid sequence of a portion of a rice acid triacylglycerol lipase derived from the nucleotide sequence of SEQ ID NO:15.

SEQ ID NO:17 is the nucleotide sequence comprising the contig assembled from the entire cDNA insert in clone ssl.pk0022.a1 and a portion of the cDNA insert in clone sdp3c.pk004.n3 encoding an entire soybean acid triacylglycerol lipase.

SEQ ID NO:18 is the deduced amino acid sequence of an entire soybean acid triacylglycerol lipase derived from the nucleotide sequence of SEQ ID NO:17.

SEQ ID NO:19 is the nucleotide sequence comprising the contig assembled from the entire cDNA insert in clone sre.pk0004.d7 and a portion of the cDNA insert in clones sls1c.pk009.o2 and srr1c.pk001.m19 encoding an entire soybean acid triacylglycerol lipase.

SEQ ID NO:20 is the deduced amino acid sequence of an entire soybean acid triacylglycerol lipase derived from the nucleotide sequence of SEQ ID NO:19.

SEQ ID NO:21 is the nucleotide sequence comprising the entire cDNA insert in clone cr1n.pk0145.c6 encoding half of a corn neutral triacylglycerol lipase.

SEQ ID NO:22 is the deduced amino acid sequence of half of a corn neutral triacylglycerol lipase derived from the nucleotide sequence of SEQ ID NO:21.

SEQ ID NO:23 is the nucleotide sequence comprising the contig assembled from a portion of the cDNA insert in clones p0010.cbpbe40r, p0083.cldcq17r, p0048.cqlac25r, p0118.chsbw59r, cr1.pk0011.c9 and cdo1c.pk002.c22 encoding an entire corn neutral triacylglycerol lipase.

SEQ ID NO:24 is the deduced amino acid sequence of an entire corn neutral triacylglycerol lipase derived from the nucleotide sequence of SEQ ID NO:23.

SEQ ID NO:25 is the nucleotide sequence comprising the contig assembled from the entire cDNA insert in clone cr1n.pk0127.h8 and a portion of the cDNA insert in clones p0037.crwan02r, p0004.cb1fm22r, p0004.cb1ei43r, cco1n.pk068.o9 and p0093.cssao39r encoding most of a corn neutral triacylglycerol lipase.

SEQ ID NO:26 is the deduced amino acid sequence of most of a corn neutral triacylglycerol lipase derived from the nucleotide sequence of SEQ ID NO:25.

SEQ ID NO:27 is the nucleotide sequence comprising a portion of the cDNA insert in clone rdr1f.pk002.f11 encoding a portion of a rice neutral triacylglycerol lipase.

SEQ ID NO:28 is the deduced amino acid sequence of a portion of a rice neutral triacylglycerol lipase derived from the nucleotide sequence of SEQ ID NO:27.

SEQ ID NO:29 is the nucleotide sequence comprising the contig assembled from the entire cDNA insert in clone sre.pk0058.b1 and a portion of the cDNA insert in clone sah1c.pk001.k20 encoding a substantial portion of a soybean neutral triacylglycerol lipase.

SEQ ID NO:30 is the deduced amino acid sequence of a substantial portion of a soybean neutral triacylglycerol lipase derived from the nucleotide sequence of SEQ ID NO:29.

SEQ ID NO:31 is the nucleotide sequence comprising the entire cDNA insert in clone sr1.pk0079.e1 encoding the C-terminal half of a soybean neutral triacylglycerol lipase.

SEQ ID NO:32 is the deduced amino acid sequence of the C-terminal half of a soybean neutral triacylglycerol lipase derived from the nucleotide sequence of SEQ ID NO:31.

SEQ ID NO:33 is the nucleotide sequence comprising the entire cDNA insert in clone wr1.pk0115.f5 encoding a portion of a wheat neutral triacylglycerol lipase.

SEQ ID NO:34 is the deduced amino acid sequence of a portion of a wheat neutral triacylglycerol lipase derived from the nucleotide sequence of SEQ ID NO:33.

SEQ ID NO:35 is the amino acid sequence of a Canis familiaris acid triacylglycerol lipase, NCBI General Identifier No. 3041702.

SEQ ID NO:36 is the amino acid sequence of a Caenorhabditis elegans acid triacylglycerol lipase, NCBI General Identifier No. 3165581.

The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. As used herein, “contig” refers to an assemblage of overlapping nucleic acid sequences to form one contiguous nucleotide sequence. For example, several DNA sequences can be compared and aligned to identify common or overlapping regions. The individual sequences can then be assembled into a single contiguous nucleotide sequence.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate alteration of gene expression by antisense or co-suppression technology or alteration of the functional properties of the resulting protein molecule. It is therefore understood that the invention encompasses more than the specific exemplary sequences.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a gene which result in the production of a chemically equivalent amino acid at a given site, but do not effect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, substantially similar nucleic acid fragments may also be characterized by their ability to hybridize, under stringent conditions (0.1×SSC, 0.1% SDS, 65° C.), with the nucleic acid fragments disclosed herein.

Substantially similar nucleic acid fragments of the instant invention may also be characterized by the percent similarity of the amino acid sequences that they encode to the amino acid sequences disclosed herein, as determined by algorithms commonly employed by those skilled in this art. Preferred are those nucleic acid fragments whose nucleotide sequences encode amino acid sequences that are 80% similar to the amino acid sequences reported herein. More preferred nucleic acid fragments encode amino acid sequences that are 90% similar to the amino acid sequences reported herein. Most preferred are nucleic acid fragments that encode amino acid sequences that are 95% similar to the amino acid sequences reported herein. Sequence alignments and percent similarity calculations were performed using the Megalign program of the LASARGENE bioinformatics computing suite (DNASTAR Inc., Madison, Wis.). Multiple alignment of the sequences was performed using the Clustal method of alignment (Higgins, D. G. and Sharp, P. M. (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool; Altschul, S. F., et al. (1993) J. Mol. Biol. 215:403-410). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the acid or the neutral triacylglycerol lipase proteins as set forth in SEQ ID NOs:2, 4, 6, 8, 10,12, 14, 16,18, 20, 22, 24, 26, 28, 30, 32 and 34. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of different lengths may have identical promoter activity.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065, incorporated herein by reference). The complementarity of an antisense RNA may be with any part of the specific gene transcript, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“Altered levels” refers to the production of gene product(s) in transgenic organisms in amounts or proportions that differ from that of normal or non-transformed organisms.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels, J. J., (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein T. M. et al. (1987) Nature (London) 327:70-73; U.S. Pat. No. 4,945,050, incorporated herein by reference).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Maniatis”).

Nucleic acid fragments encoding at least a portion of several triacylglycerol lipases have been isolated and identified by comparison of random plant cDNA sequences to public databases containing nucleotide and protein sequences using the BLAST algorithms well known to those skilled in the art. Table 1 lists the proteins that are described herein, and the designation of the cDNA clones that comprise the nucleic acid fragments encoding these proteins.

TABLE 1
Triacylglycerol Lipases

| Enzyme | Clone | Plant |
|---|---|---|
| Triacylglycerol | cen3n.pk0129.e9 | Corn |
| Acid Lipase | Contig of: | Corn |
| | p0075.cslag33r | |
| | p0126.cnlay46r | |
| | p0014.ctuty54r | |
| | p0102.ceral64r | Corn |
| | p0126.cnlcm37r | Corn |
| | ncs.pk0013.h1 | Catalpa |
| | rlr72.pk0015.b2 | Rice |
| | rsl1n.pk012.h7 | Rice |
| | Contig of: | Soybean |
| | sdp3c.pk004.n3 | |
| | ssl.pk0022.a1 | |
| | Contig of: | Soybean |
| | sls1c.pk009.o2 | |
| | srr1c.pk001.m19 | |
| | sre.pk0004.d7 | |
| Triacylglycerol | cr1n.pk0145.c6 | Corn |
| Neutral Lipase | Contig of: | Corn |
| | p0010.cbpbe40r | |
| | p0083.cldcq17r | |
| | p0048.cqlac25r | |
| | p0118.chsbw59r | |
| | cr1.pk0011.c9 | |
| | cdo1c.pk002.c22 | |
| | Contig of: | Corn |
| | p0037.crwan02r | |
| | p0004.cb1fm22r | |
| | p0004.cb1ei43r | |
| | cco1n.pk068.o9 | |
| | p0093.cssao39r | |
| | cr1n.pk0127.h8 | |
| | rdr1f.pk002.f11 | Rice |
| | Contig of: | Soybean |
| | sah1c.pk001.k20□ | |
| | sre.pk0058.b1 | |
| | sr1.pk0079.e1 | Soybean |
| | wr1.pk0115.f5 | Wheat |

The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other acid triacylglycerol lipases, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673; Loh et al. (1989) Science 243:217). Products generated by the 3′ and 5′ RACE procedures can be combined to generate full-length cDNAs (Frohman, M. A. and Martin, G. R., (1989) Techniques 1:165).

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, R. A. (1984) Adv. Immunol. 36:1; Maniatis).

The nucleic acid fragments of the instant invention may be used to create transgenic plants in which the disclosed acid or neutral triacylglycerol lipases are present at higher or lower levels than normal or in cell types or developmental stages in which they are not normally found. This would have the effect of altering the level of triacylglycerol and cholesteryl esters in those cells. Accumulation of fatty acids with unusual structures may be a positive phenotype in plants used for foods. Triacylglycerol lipases may also be useful in processing of plant seed oils and the development of novel seed oils.

Overexpression of the acid or the neutral triacylglycerol lipases of the instant invention may be accomplished by first constructing a chimeric gene in which the coding region is operably linked to a promoter capable of directing expression of a gene in the desired tissues at the desired stage of development. For reasons of convenience, the chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant chimeric gene can then constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant triacylglycerol lipase to different cellular compartments, or to facilitate its secretion from the cell. It is thus envisioned that the chimeric gene described above may be further supplemented by altering the coding sequence to encode a acid triacylglycerol lipase with appropriate intracellular targeting sequences such as transit sequences (Keegstra, K. (1989) Cell 56:247-253), signal sequences or sequences encoding endoplasmic reticulum localization (Chrispeels, J. J., (1991) Ann. Rev. Plant Phys. Plant Mol. Biol. 42:21-53), or nuclear localization signals (Raikhel, N. (1992) Plant Phys. 100:1627-1632) added and/or with targeting sequences that are already present removed. While the references cited give examples of each of these, the list is not exhaustive and more targeting signals of utility may be discovered in the future.

It may also be desirable to reduce or eliminate expression of genes encoding acid or neutral triacylglycerol lipases in plants for some applications. In order to accomplish this, a chimeric gene designed for co-suppression of the instant triacylglycerol lipase can be constructed by linking a gene or gene fragment encoding an acid or a neutral triacylglycerol lipase to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

The instant acid or neutral triacylglycerol lipases (or portions thereof) may be produced in heterologous host cells, particularly in the cells of microbial hosts, and can be used to prepare antibodies to the these proteins by methods well known to those skilled in the art. The antibodies are useful for detecting acid or neutral triacylglycerol lipases in situ in cells or in vitro in cell extracts. Preferred heterologous host cells for production of the instant acid or neutral triacylglycerol lipases are microbial hosts. Microbial expression systems and expression vectors containing regulatory sequences that direct high level expression of foreign proteins are well known to those skilled in the art. Any of these could be used to construct a chimeric gene for production of the instant acid or neutral triacylglycerol lipase. This chimeric gene could then be introduced into appropriate microorganisms via transformation to provide high level expression of the encoded triacylglycerol lipase. An example of a vector for high level expression of the instant acid or neutral triacylglycerol lipase in a bacterial host is provided (Example 7).

All or a substantial portion of the nucleic acid fragments of the instant invention may also be used as probes for genetically and physically mapping the genes that they are a part of, and as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al. (1980) Am. J. Hum. Genet. 32:314-331).

The production and use of plant gene-derived probes for use in genetic mapping is described in R. Bernatzky, R. and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel, J. D., et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask, B. J. (1991) Trends Genet. 7:149-154). Although current methods of FISH mapping favor use of large clones (several to several hundred KB; see Laan, M. et al. (1995) Genome Research 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian, H. H. (1989) J. Lab. Clin. Med. 114(2):95-96), polymorphism of PCR-amplified fragments (CAPS; Sheffield, V. C. et al. (1993) Genomics 16:325-332), allele-specific ligation (Landegren, U. et al. (1988) Science 241:1077-1080), nucleotide extension reactions (Sokolov, B. P. (1990) Nucleic Acid Res. 18:3671), Radiation Hybrid Mapping (Walter, M. A. et al. (1997) Nature Genetics 7:22-28) and Happy Mapping (Dear, P. H. and Cook, P. R. (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer, (1989) Proc. Natl. Acad. Sci USA 86:9402; Koes et al. (1995) Proc. Natl. Acad. Sci USA 92:8149; Bensen et al. (1995) Plant Cell 7:75). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the acid or the neutral triacylglycerol lipase. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding an acid or a neutral triacylglycerol lipase can be identified and obtained. This mutant plant can then be used to determine or confirm the natural function of the acid or the neutral triacylglycerol lipase gene product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Composition of cDNA Libraries; Isolation and Sequencing of cDNA Clones

cDNA libraries representing mRNAs from various Catalpa, corn, rice, soybean and wheat tissues were prepared. The characteristics of the libraries are described below.

TABLE 2
cDNA Libraries from Catalpa, Corn, Rice, Soybean and Wheat

| Library | Tissue | Clone |
|---|---|---|
| cco1n | Corn Cob of 67 Day Old Plants | cco1n.pk068.o9 |
| | Grown in Green House* | |
| cdo1c | Corn Ovary (including pedicel and | cdo1c.pk002.c22 |
| | glumes), 5 Days After Silking | |
| cen3n | Corn Endosperm 20 Days After | cen3n.pk0129.e9 |
| | Pollination* | |
| cr1 | Corn Root From 7 Day Old | cr1.pk0011.c9 |
| | Seedlings | |
| cr1n | Corn Root From 7 Day Old | cr1n.pk0127.h8 |
| | Seedlings* | cr1n.pk0145.c6 |
| ncs | Catalpa speciosa Developing Seed | ncs.pk0013.h1 |
| p0004 | Corn Immature Ear | p0004.cb1ei43r |
| p0010 | Corn Log Phase Suspension Cells | p0004.cb1fm22r |
| | Treated With A23187** | p0010.cbpbe40r |
| p0014 | Corn Leaves 7 and 8 From 3 Foot- | p0014.ctuty54r |
| | Tall Plant | |
| p0037 | Corn V5 Stage Roots Infested With | p0037.crwan02r |
| | Corn Root Worm | |
| p0048 | Corn Embryo (Axis and Scuttelum) | p0048.cqlac25r |
| | One Day After Germination | |
| p0075 | Corn Shoot And Leaf Material From | p0075.cslag33r |
| | Dark-Grown 7 Day-Old Seedlings | |
| p0083 | Corn Whole Kernels 7 Days After | p0083.cldcq17r |
| | Pollination | |
| p0093 | Corn Stalk And Shank, 2-3 Weeks | p0093.cssao39r |
| | After Pollen Shed* | |
| p0102 | Corn Early Meiosis Tassels* | p0102.ceral64r |
| p0118 | Corn Stem Tissue Pooled From the 4 | p0118.chsbw59r |
| | to 5 Internodes Subtending The | |
| | Tassel At Stages V8-V12, Night | |
| | Harvested* | |
| p0126 | Corn Leaf Tissue From V8-V10 | p0126.cnlay46r |
| | Stages, Pooled, Night-Harvested | p0126.cnlcm37r |
| rdr1f | Rice Developing Root of 10 Day | rdr1f.pk002.f11 |
| | Old Plant | |
| rlr72 | Rice Leaf 15 Days After Germination, | rlr72.pk0015.b2 |
| | 72 Hours After Infection of Strain | |
| | Magaporthe grisea 4360-R-62 | |
| | (AVR2-YAMO); Resistant | |
| rsl1n | Rice 15-Day-Old Seedling* | rsl1n.pk012.h7 |
| sah1c | Soybean Sprayed With Authority ™ | sah1c.pk001.k20 |
| | Herbicide | |
| sdp3c | Soybean Developing Pods (8-9 mm) | sdp3c.pk004.n3 |
| sls1c | Soybean Infected With Sclerotinia | sls1c.pk009.o2 |
| | sclerotiorum Mycelium | |
| sr1 | Soybean Root | sr1.pk0079.e1 |
| sre | Soybean Root Elongation Zone 4 to | sre.pk0004.d7 |
| | 5 Days After Germination | sre.pk0058.b1 |
| srr1c | Soybean 8-Day-Old Root | srr1c.pk001.m19 |
| ssl | Soybean Seedling 5-10 Days | ssl.pk0022.a1 |
| | After Germination | |
| wr1 | Wheat Root From 7 Day Old Seedling | wr1.pk0115.f5 |

*These libraries were normalized essentially as described in U.S. Pat. No. 5,482,845
**A23187 is commercially available from several sources including Calbiochem.

cDNA libraries were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al. (1991) Science 252:1651). The resulting ESTs were analyzed using a Perkin Elmer Model 377 fluorescent sequencer.

### Example 2

### Identification of cDNA Clones

ESTs encoding triacylglycerol lipases were identified by conducting BLAST (Basic Local Alignment Search Tool; Altschul, S. F., et al. (1993) J. Mol. Biol. 215:403-410) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

### Example 3

### Characterization of cDNA Clones Encoding Acid Triacylglycerol Lipases

The BLASTX search using the EST sequences from clones cen3n.pk0129.e9, ncs.pk0013.h1, a contig sequence assembled from the EST sequences from clones rlr72.pk0015.b2 and rr1.pk0051.f10, a contig sequence assembled from the EST sequences of clones ssl.pk0022.a1 and sr1.pk0098.b11, and a contig sequence assembled from the EST sequences from clones sre.pk0004.d7 and sre.pk0001.b2 revealed similarity of the proteins encoded by the cDNAs and the contigs to acid triacylglycerol lipases from human and rat (GenBank Accession Nos. are listed below). The BLAST results for each of these ESTs and contigs are shown in Table 3:

TABLE 3
BLAST Results for Clones Encoding Polypeptides
Homologous to Acid Triacylglycerol Lipases

| | | | BLAST |
|---|---|---|---|
| | | GenBank | pLog |
| Clone | Organism | Accession No. | Score |
| cen3n.pk0129.e9 | Human | X05997 | 14.52 |
| ncs.pk0013.h1 | Rat | X02309 | 14.70 |
| Contig of | Human | U08464 | 16.40 |
| rlr72.pk0015.b2 | | | |
| rr1.pk0051.f10 | | | |
| Contig of | Rat | X02309 | 15.22 |
| ssl.pk0022.a1 | | | |
| sr1.pk0098.b11 | | | |
| Contig of | Human | X76488 | 22.00 |
| sre.pk0004.d7 | | | |
| sre.pk0001.b2 | | | |

TBLASTN analysis of the proprietary plant EST database indicated that other corn, rice and soybean sequences also encoded acid triacylglycerol lipases. The BLASTX search using the contig sequences assembled with the EST sequences from clones p0075.cslag33r, p0126.cnlay46r and p0014.ctuty54r revealed similarity of the proteins encoded by the cDNAs to acid triacylglycerol lipase from Homo sapiens (NCBI General Identifier No. 505053). The BLASTX search using the EST sequences from clones p0102.ceral64r and using the contig sequences assembled from the entire cDNA insert in clone ssl.pk0022.a1 and the EST sequences from clone sdp3c.pk004.n3 revealed similarity of the proteins encoded by the cDNAs to acid triacylglycerol lipase from Canis familiaris (NCBI General Identifier No. 3041702). The BLASTX search using the EST sequences from clone p0126.cnlcm37r revealed similarity of the proteins encoded by the cDNAs to Drosophila melanogaster (NCBI General Identifier No. 2894442). The BLASTX search using the EST sequences from clone rsl1n.pk012.h7 revealed similarity of the proteins encoded by the cDNAs to acid triacylglycerol lipase from Rattus norvegicus (NCBI General Identifier No. 126307). The BLAST results for each of these sequences is shown in Table 4:

TABLE 4
BLAST Results for Clones Encoding Polypeptides
Homologous to Acid Triacylglycerol Lipase

| | | BLAST |
|---|---|---|
| | NCBI General | pLog |
| Clone | Identifier No. | Score |
| Contig of: | 505053 | 35.00 |
| p0075.cslag33r | | |
| p0126.cnlay46r | | |
| p0014.ctuty54r | | |
| p0102.ceral64r | 3041702 | 11.30 |
| p0126.cnlcm37r | 2894442 | 10.40 |
| rsl1n.pk012.h7 | 126307 | 7.00 |

The sequence of the entire cDNA insert in clone cen3n.pk0129.e9 was determined and is shown in SEQ ID NO:1; the deduced amino acid sequence of this cDNA is shown in SEQ ID NO:2. The amino acid sequence set forth in SEQ ID NO:2 was evaluated by BLASTP, yielding a pLog value of 15.00 versus the Homo sapiens sequence (NCBI General Identifier No. 126306). The sequence of the 3′-terminal portion from clone ncs.pk0013.h1 is shown in SEQ ID NO:3; the deduced amino acid sequence of this cDNA is shown in SEQ ID NO:4. The sequence of the 5′-terminal portion from clone ncs.pk0013.h1 is shown in SEQ ID NO:5; the deduced amino acid sequence of this cDNA is shown in SEQ ID NO:6. The sequence of the contig assembled from the EST sequences from clones p0075.cslag33r, p0126.cnlay46r and p0014.ctuty54r is shown in SEQ ID NO:7, the deduced amino acid sequence of this cDNA is shown in SEQ ID NO:8. The sequence of a portion of the cDNA insert from clone p0102.ceral64r is shown in SEQ ID NO:9; the deduced amino acid sequence of this cDNA is shown in SEQ ID NO:10. The sequence of a portion of the cDNA insert from clone p0126.cnlcm37r is shown in SEQ ID NO:11; the deduced amino acid sequence of this cDNA is shown in SEQ ID NO:12. The sequence of the entire cDNA insert in clone rlr72.pk0015.b2 was determined and is shown in SEQ ID NO:13; the deduced amino acid sequence of this cDNA is shown in SEQ ID NO:14. The amino acid sequence set forth in SEQ ID NO:14 was evaluated by BLASTP, yielding a pLog value of 53.30 versus the C. elegans sequence (NCBI General Identifier No. 3165581). The sequence of a portion of the cDNA insert from clone rsl1n.pk012.h7 is shown in SEQ ID NO:15; the deduced amino acid sequence of this cDNA is shown in SEQ ID NO:16. The sequence of the entire cDNA insert in clone ssl.pk0022.a1 was determined and a contig assembled with this sequence and the EST sequences from clone sdp3c.pk004.n3. The sequence of this contig is shown in SEQ ID NO:17; the deduced amino acid sequence of this cDNA is shown in SEQ ID NO:18. The amino acid sequence set forth in SEQ ID NO:18 was evaluated by BLASTP, yielding a pLog value of 59.40 versus the C. familiaris sequence (NCBI General Identifier No. 3041702). The sequence of the entire cDNA insert in clone sre.pk0004.d7 was determined and a contig assembled with this sequence and the EST sequences from clones sls1c.pk009.o2 and srr1c.pk001.m19. The sequence of this contig is shown in SEQ ID NO:19; the deduced amino acid sequence of this cDNA is shown in SEQ ID NO:20. The amino acid sequence set forth in SEQ ID NO:20 was evaluated by BLASTP, yielding a pLog value of 48.70 versus the C. elegans sequence (NCBI General Identifier No. 3165581).

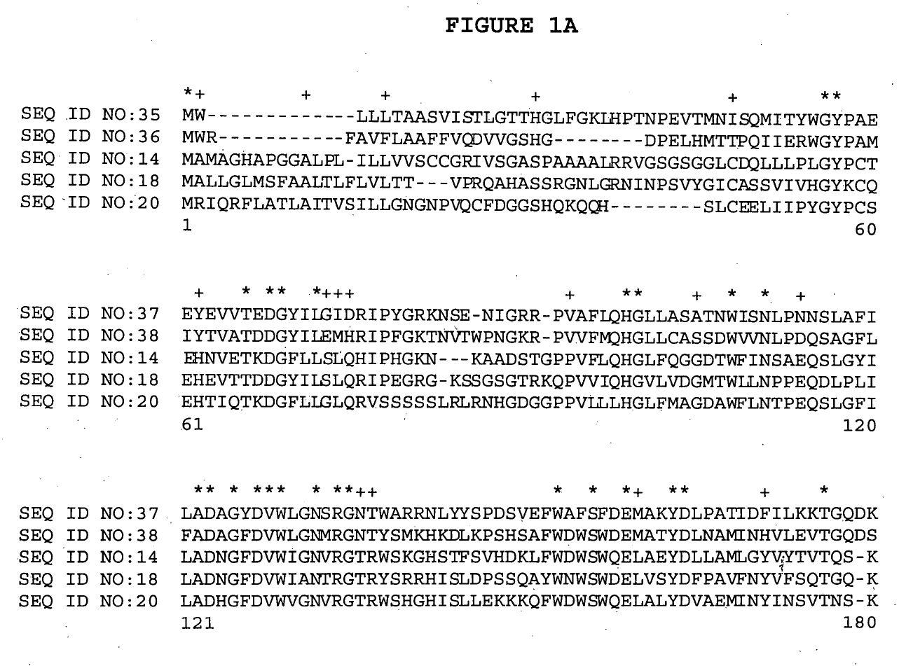

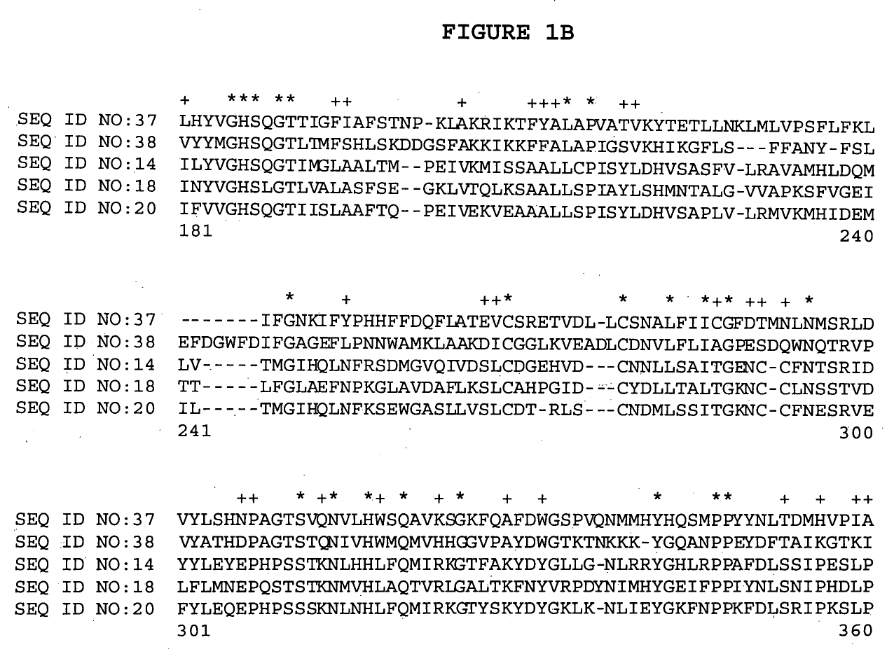

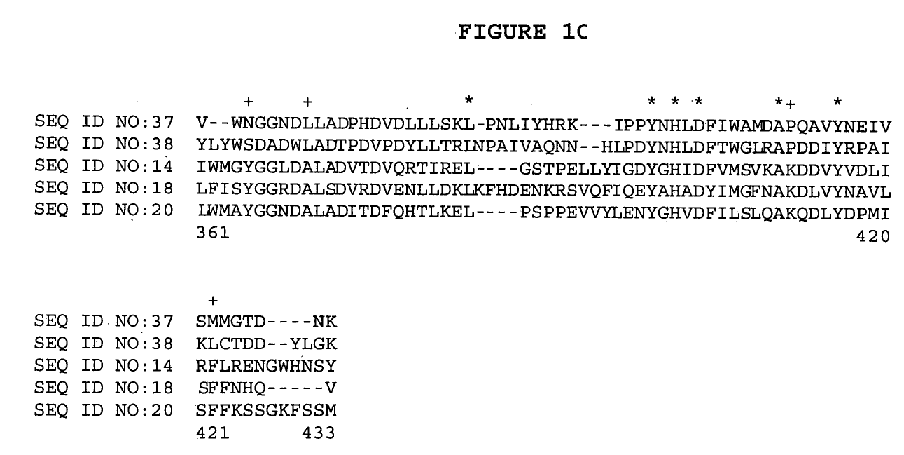

FIGS. 1A-C presents an alignment of the amino acid sequences set forth in SEQ ID NOS:14, 18 and 20 with the Canis familiaris sequence (NCBI General Identifier No. 3041702; SEQ ID NO:35) and the Caenorhabditis elegans sequence (NCBI General Identifier No. 3165581; SEQ ID NO:36). The data in Table 5 presents a calculation of the percent similarity of the amino acid sequences set forth in SEQ ID NOS:2, 4, 6, 8,10, 12, 14, 18 and 20 and the Caenorhabditis elegans sequence.

TABLE 5
Percent Similarity of Amino Acid Sequences Deduced From
the Nucleotide Sequences of cDNA Clones Encoding Polypeptides
Homologous to Acid Triacylglycerol Lipase

| | |
|---|---|
| SEQ | Percent Identity to |

| Clone | ID NO. | 3041702 | 3165581 |
|---|---|---|---|
| cen3n.pk0129.e9:fis | 2 | 27.1 | 22.9 |
| ncs.pk0013.h1.fis1 | 4 | 27.4 | 21.4 |
| ncs.pk0013.h1.fis2 | 6 | 30.6 | 29.9 |
| p0075.cslag33r | 8 | 22.0 | 23.1 |
| p0126.cnlay46r | | | |
| p0014.ctuty54r | | | |
| p0102.ceral64r | 10 | 28.8 | 22.4 |
| p0126.cnlcm37r | 12 | 26.7 | 22.2 |
| rlr72.pk0015.b2:fis | 14 | 24.9 | 25.6 |
| rsl1n.pk012.h7 | 16 | 22.5 | 22.5 |
| sdp3c.pk004.n3 | 18 | 27.4 | 23.1 |
| ssl.pk0022.a1.fis1 | | | |
| sls1c.pk009.o2 | 20 | 23.1 | 24.8 |
| srr1c.pk001.m19 | | | |
| sre.pk0004.d7.fis1 | | | |

Sequence alignments and percent similarity calculations were performed using the Megalign program of the LASARGENE bioinformatics computing suite (DNASTAR Inc., Madison, Wis.). Multiple alignment of the amino acid sequences was performed using the Clustal method of alignment (Higgins, D. G. and Sharp, P. M. (1989) CABIOS. 5:151-153) with the default parameters (GAP PENALTY=10, GAP LENGTH PENALTY=10).

Sequence alignments and BLAST scores and probabilities indicate that the instant nucleic acid fragments encode an entire rice acid triacylglycerol lipase, two different entire soybean acid triacylglycerol lipases, portions from several different corn acid triacylglycerol lipases, portions of a Catalpa acid triacylglycerol lipase and a portion of a rice acid triacylglycerol lipase. These sequences represent the first plant sequences encoding acid triacylglycerol lipases.

### Example 4

### Characterization of cDNA Clones Encoding Neutral Triacylglycerol Lipases

The BLASTX search using the contig sequence assembled from the EST sequences from clones cr1n.pk0127.h8 and cr1n.pk0134.d3, and EST sequences from clones cr1n.pk0145.c6, s1.03b01, se3.01a04, sfl1.pk0049.d11, sr1.pk0079.e1, sr1.pk0030.g8, sre.pk0058.b1, wl1n.pk0014.e10, wl1n.pk0038.e3 and wr1.pk0115.f5 revealed similarity of the proteins encoded by the contig and the cDNAs to neutral triacylglycerol lipases from several organisms. Table 6 shows the BLAST results for the contig and each of the ESTs, the NCBI database accession number, and the organism the closest art sequence is derived from:

TABLE 6
BLAST Results for Clones Encoding Polypeptides
Homologous to Neutral Triacylglycerol Lipases

| | | NCBI | BLAST |
|---|---|---|---|
| | | Accession | pLog |
| Clone | Organism | No. | Score |
| Contig of: | Thermomyces | 999873 | 10.00 |
| cr1n.pk0127.h8 | lanuginosus | | |
| cr1n.pk0134.d3 | | | |
| cr1n.pk0145.c6 | Caenorhabditis | 927399 | 8.70 |
| | elegans | | |
| sr1.pk0079.e1 | Rhizopus | 251079 | 6.70 |
| | niveus | | |
| sre.pk0058.b1 | Rhizomucor | 417256 | 8.10 |
| | miehei | | |
| wr1.pk0115.f5 | Rhizomucor | 82777 | 6.00 |
| | miehei | | |

TBLASTN analysis of the proprietary plant EST database indicated that rice clones as well as other corn and soybean clones also encode neutral triacylglycerol lipases. The BLASTX search using the contig sequences assembled from clones p0010.cbpbe40r, p0083.cldcq17r, p0048.cqlac25r, p0118.chsbw59r, cr1.pk0011.c9 and cdo1c.pk002.c22 and using the EST sequences from clone rdr1f.pk002.f11 revealed similarity of the proteins encoded by the cDNAs to neutral triacylglycerol lipase from C. elegans (NCBI General Identifier No. 3877256). The BLAST results for each of these sequences are shown in Table 7:

TABLE 7
BLAST Results for Clones Encoding Polypeptides
Homologous to Neutral Triacylglycerol Lipases

| | | NCBI | |
|---|---|---|---|
| | | General | BLAST |
| | | Identifier | pLog |
| Clone | Organism | No. | Score |
| cr1n.pk0145.c6 | Caenorhabditis | 3877256 | 9.30 |
| | elegans | | |
| Contig of: | Caenorhabditis | 3877256 | 18.40 |
| p0010.cbpbe40r | elegans | | |
| p0083.cldcq17r | | | |
| p0048.cqlac25r | | | |
| p0118.chsbw59r | | | |
| cr1.pk0011.c9 | | | |
| cdo1c.pk002.c22 | | | |
| Contig of: | Thermomyces | 2997733 | 6.15 |
| p0037.crwan02r | lanuginosus | | |
| p0004.cb1fm22r | | | |
| p0004.cb1ei43r | | | |
| cco1n.pk068.o9 | | | |
| p0093.cssao39r | | | |
| cr1n.pk0127.h8 | | | |
| rdr1f.pk002.f11 | Caenorhabditis | 3877256 | 10.22 |
| | elegans | | |
| Contig of: | Rhizomucor | 417256 | 6.22 |
| sah1c.pk001.k20 | miehei | | |
| □ sre.pk0058.b1 | | | |
| sr1.pk0079.e1 | Rhizopus | 3299795 | 5.70 |
| | niveus | | |
| wr1.pk0115.f5 | Caenorhabditis | 3877256 | 14.00 |
| | elegans | | |

The sequence of the entire cDNA insert in clone cr1n.pk0145.c6 was determined and is shown in SEQ ID NO:21; the deduced amino acid sequence of this cDNA is shown in SEQ ID NO:22. The amino acid sequence set forth in SEQ ID NO:22 was evaluated by BLASTP, yielding a pLog value of 10.70 versus the C. elegans sequence. The sequence of the contig assembled from a portion of the cDNA insert in clones p0010.cbpbe40r, p0083.cldcq17r, p0048.cqlac25r, p0118.chsbw59r, cr1.pk0011.c9 and cdo1c.pk002.c22 is shown in SEQ ID NO:23; the deduced amino acid sequence of this cDNA is shown in SEQ ID NO:24. The sequence of the entire cDNA insert in clone cr1n.pk0127.h8 was determined and a contig assembled with this sequence and the sequence from a portion of the cDNA insert in clones p0037.crwan02r, p0004.cb1fm22r, p0004.cb1ei43r, cco1n.pk068.o9 and p0093.cssao39r. The sequence of this contig is shown in SEQ ID NO:25; the deduced amino acid sequence of this cDNA is shown in SEQ ID NO:26. The amino acid sequence set forth in SEQ ID NO:26 was evaluated by BLASTP, yielding a pLog value of 9.70 versus the Thermomyces lanuginosus sequence. The sequence of a portion of the cDNA insert from clone rdr1f.pk002.f11 is shown in SEQ ID NO:27; the deduced amino acid sequence of this cDNA is shown in SEQ ID NO:28. The sequence of the entire cDNA insert in clone sre.pk0058.b1 was determined and a contig assembled with this sequence and the sequence of a portion of the cDNA insert in clone sah1c.pk001.k20. The sequence of this contig is shown in SEQ ID NO:29; the deduced amino acid sequence of this cDNA is shown in SEQ ID NO:30. The amino acid sequence set forth in SEQ ID NO:30 was evaluated by BLASTP, yielding a pLog value of 8.05 versus the Rhizomucor miehei sequence. The sequence of the entire cDNA insert in clone sr1.pk0079.e1 was determined and is shown in SEQ ID NO:31; the deduced amino acid sequence of this cDNA is shown in SEQ ID NO:32. The amino acid sequence set forth in SEQ ID NO:32 was evaluated by BLASTP, yielding a pLog value of 7.52 versus the Rhizopus niveus sequence. The sequence of the entire cDNA insert in clone wr1.pk0115.f5 was determined and is shown in SEQ ID NO:33; the deduced amino acid sequence of this cDNA is shown in SEQ ID NO:34. The amino acid sequence set forth in SEQ ID NO:34 was evaluated by BLASTP, yielding a pLog value of 13.52 versus the Caenorhabditis elegans sequence.

The data in Table 8 presents a calculation of the percent similarity of the amino acid sequences set forth in SEQ ID NOS:22, 24, 26, 28, 30, 32 and 34 and the Caenorhabditis elegans, Rhizomucor miehei and Thermomyces lanuginosus sequences.

TABLE 8
Percent Similarity of Amino Acid Sequences Deduced From
the Nucleotide Sequences of cDNA Clones Encoding Polypeptides
Homologous to Neutral Triacylglycerol Lipase

| | SEQ | Percent Similarity to | | |
|---|---|---|---|---|
| Clone | ID NO. | 3877256 | 2997733 | 417256 |
| cr1n.pk0145.c6 | 22 | 15.1 | 13.2 | 16.8 |
| Contig of: | 24 | 60.5 | 17.5 | 22.9 |
| p0010.cbpbe40r | | | | |
| p0083.cldcq17r | | | | |
| p0048.cqlac25r | | | | |
| p0118.chsbw59r | | | | |
| cr1.pk0011.c9 | | | | |
| cdo1c.pk002.c22 | | | | |
| Contig of: | 26 | 18.5 | 14.3 | 15.1 |
| p0037.crwan02r | | | | |
| p0004.cb1fm22r | | | | |
| p0004.cb1ei43r | | | | |
| cco1n.pk068.o9 | | | | |
| p0093.cssao39r | | | | |
| cr1n.pk0127.h8 | | | | |
| rdr1f.pk002.f11 | 28 | 12.6 | 20.6 | 22.9 |
| Contig of: | 32 | 15.1 | 10.5 | 17.0 |
| sah1c.pk001.k20 | | | | |
| □ sre.pk0058.b1 | | | | |
| sr1.pk0079.e1 | 34 | 14.3 | 21.1 | 24.6 |
| wr1.pk0115.f5 | | 37.0 | 22.0 | 26.0 |

Sequence alignments and percent similarity calculations were performed using the Megalign program of the LASARGENE bioinformatics computing suite (DNASTAR Inc., Madison, Wis.). Multiple alignment of the amino acid sequences was performed using the Clustal method of alignment (Higgins, D. G. and Sharp, P. M. (1989) CABIOS. 5:151-153) with the default parameters (GAP PENALTY=10, GAP LENGTH PENALTY=10).

Sequence alignments and BLAST scores and probabilities indicate that the instant nucleic acid fragments encode three different corn neutral triacylglycerol lipases(one portion and two entire or nearly entire), two different soybean triacylglycerol lipases (one portion and one nearly entire) and a portion of a wheat triacylglycerol lipase. These sequences represent the first monocot and soybean sequences encoding neutral triacylglycerol lipases.

### Example 5

### Expression of Chimeric Genes in Monocot Cells

A chimeric gene comprising a cDNA encoding triacylglycerol lipases in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (Nco I or Sma I) can be incorporated into the oligonucleotides to provide proper orientation of the DNA fragment when inserted into the digested vector pML103 as described below. Amplification is then performed in a standard PCR. The amplified DNA is then digested with restriction enzymes Nco I and Sma I and fractionated on an agarose gel. The appropriate band can be isolated from the gel and combined with a 4.9 kb Nco I-Sma I fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb Sal I-Nco I promoter fragment of the maize 27 kD zein gene and a 0.96 kb Sma I-Sal I fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding a triacylglycerol lipase, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein, T. M. et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kaptonμ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 6

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant triacylglycerol lipase in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embroys may then be transformed with the expression vector comprising sequences encoding a triacylglycerol lipase. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein T. M. et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the triacylglycerol lipase and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 7

### Expression of Chimeric Genes in Microbial Cells

The cDNAs encoding the instant triacylglycerol lipases can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG™ low melting agarose gel (FMC). Buffer and agarose contain 10 μg/ml ethidium bromide for visualization of the DNA fragment. The fragment can then be purified from the agarose gel by digestion with GELase™ (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, also referred to as NEB, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the triacylglycerol lipase are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

What is claimed is:

1. An isolated nucleic acid fragment encoding all or a substantial portion of an acid triacylglycerol lipase comprising a member selected from the group consisting of:
(a) an isolated nucleic acid fragment encoding all or a substantial portion of the amino acid sequence set forth in a member selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18 and SEQ ID NO:20;
(b) an isolated nucleic acid fragment that is substantially similar to an isolated nucleic acid fragment encoding all or a substantial portion of the amino acid sequence set forth in a member selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18 and SEQ ID NO:20; and
(c) an isolated nucleic acid fragment that is complementary to (a) or (b).

2. An isolated polynucleotide comprising:
(a) a nucleotide sequence encoding a polypeptide having triacylglycerol lipase activity, wherein the polypeptide has an amino acid sequence of at least 80% sequence identity, based on the Clustal V method of alignment, when compared to one of SEQ ID NO:24, or
(b) a full complement of the nucleotide sequence.

3. The polynucleotide of claim 2, wherein the amino acid sequence of the polypeptide has at least 85% sequence identity, based on the Clustal V method of alignment, when compared to SEQ ID NO:24.

4. The polynucleotide of claim 2, wherein the amino acid sequence of the polypeptide has at least 90% sequence identity, based on the Clustal V method of alignment, when compared to SEQ ID NO:24.

5. The polynucleotide of claim 2, wherein the amino acid sequence of the polypeptide has at least 95% sequence identity, based on the Clustal V method of alignment, when compared to SEQ ID NO:24.

6. The polynucleotide of claim 2, wherein the amino acid sequence of the polypeptide comprises SEQ ID NO:24.

7. The polynucleotide of claim 2 wherein the nucleotide sequence comprises SEQ ID NO:23.

8. A vector comprising the polynucleotide of claim 2.

9. A recombinant DNA construct comprising the polynucleotide of claim 2 operably linked to at least one regulatory sequence.

10. A method for transforming a cell, comprising transforming a cell with the polynucleotide of claim 2.

11. A cell comprising the recombinant DNA construct of claim 9.

12. A method for producing a plant comprising transforming a plant cell with the polynucleotide of claim 2 and regenerating a plant from the transformed plant cell.

13. A plant comprising the recombinant DNA construct of claim 9.

14. A seed comprising the recombinant DNA construct of claim 9.

15. A method for isolating a polypeptide having triacylglycerol lipase activity comprising isolating the polypeptide from a cell or culture medium of the cell, wherein the cell comprises a recombinant DNA construct comprising the polynucleotide of claim 2 operably linked to at least one regulatory sequence.

16. A method of altering the level of triacylglycerol and cholesteryl esters in a host cell comprising: (a) transforming a host cell with the recombinant DNA construct of claim 9; and (b) growing the transformed host cell under conditions that are suitable for expression of the recombinant DNA construct wherein expression of the recombinant DNA construct results in production of altered levels of the triacylglycerol and cholesteryl esters in the transformed host cell when compared to a non-transformed cell.

17. A method of obtaining novel plant seed oils comprising:
a) transforming a plant cell with the recombinant DNA construct of claim 9;
b) regenerating a transgenic plant from said plant cell;
c) allowing the transgenic plant to set seed;
d) harvesting seed from said transgenic plant;
d) isolating seed oil from said seed; and
d) comparing the seed oil isolated from the seed from said transgenic plant with seed oil isolated from a non-transgenic plant
wherein said seed oil isolated from the seed from said transgenic plant is novel when compared to said seed oil isolated from a non-transgenic plant.
